(19) United States
(12) Patent Application Publication
(10) Pub. No.: US 2025/0329464 A1
(43) Pub. Date: Oct. 23, 2025

(54) INDUCTIVE GRAPH MACHINE LEARNING METHOD AND SYSTEM

(71) Applicant: NEC Laboratories Europe GmbH, Heidelberg (DE)

(72) Inventors: Federico ERRICA, Heidelberg (DE); Giampaolo PILEGGI, Heidelberg (DE)

(21) Appl. No.: 18/870,704

(22) PCT Filed: Nov. 14, 2022
(86) PCT No.: PCT/EP2022/081838

§ 371 Date: Dec. 2, 2024

(30) Foreign Application Priority Data
Sep. 12, 2022 (EP) 22195185.8

(51) Int. Cl. G16H 50/30

(52) U.S. Cl. CPC G16H 50/30

(57) ABSTRACT

A method of inductive graph machine learning uses information recorded or collected from an established network including a plurality of entities with relationships existing between the plurality of entities to generate a graph representation of the established network. The plurality of entities form nodes and the relationships existing between the plurality of entities form edges of the graph representation. A new entity is mapped to a latent space. An extended network is created by connecting the new entity to one or more of the plurality of entities of the established network according to their distance in the latent space. The extended network and a graph machine learning (ML) predictor is optimized and used to make predictions about the new entity.

### CROSS REFERENCE TO RELATED APPLICATIONS

This application is a U.S. National Phase application under 35 U.S.C. § 371 of International Application No. PCT/EP2022/081838, filed on Nov. 14, 2022, and claims benefit to European Patent Application No. 22195185.8, filed on Sep. 12, 2022. The International Application was published in English on Mar. 21, 2024 as WO 2024/056201 A1 under PCT Article 21 (2).

### FIELD

The present invention relates to computer-implemented methods and systems of inductive graph machine learning.

### BACKGROUND

In the vast majority of practical scenarios, machine learning (ML) systems are trained on a fixed amount of training samples that are provided beforehand. At inference time, classical ML predictors take the input vector corresponding to the new sample and produce the required output thanks to the assumptions that samples are independent and identically distributed (i.i.d.).

The situation becomes far more challenging when the input is structured as a graph (e.g., a community), with entities interacting with each other. In this case, graph ML methods typically predict the values of interest for the entities based on a connectivity structure provided in advance. However, when a new entity arrives and is supposed to join the known graph, there may not exist a programmable algorithm to reliably connect the new entity to existing ones. This is the case, for instance, for social networks, where the users freely choose to connect to each other without a pre-defined schema. When a user joins a new network, a graph ML predictor may struggle to generalize well until a sufficient number of connections has been established. On the other hand, in a given patient network, for instance, patients may have been connected together depending on information not available when a new patient arrives at the hospital. Assuming that such a network helps in predicting the clinical risk of patients, it would be helpful to have an approximate way to connect the new patient to others before making a prediction.

Being able to handle new entities has repercussions on the developed methods, which cannot be transductive as in the case of Knowledge Graph (KG) methods; transductive methods assume that it is possible to associate a unique identifier to each entity of the KG, but they cannot generalize to unseen entities. In the literature, a plethora of inductive methods have been developed for many different tasks, but almost none of them try to take into account the problem of connecting new entities to an initial graph in order to make a prediction. To do so, structure-reconstruction loss functions are usually employed.

In Bojchevski, Aleksandar, and Stephan Günnemann. “Deep Gaussian Embedding of Graphs: Unsupervised Inductive Learning via Ranking.” International Conference on Learning Representations. 2018, training is carried out using the known structure but the inference phase does not require the structure at all.

A similar structure-reconstruction loss is exploited in Salehi, Amin, and Hasan Davulcu. “Graph Attention Auto-Encoders.” 2020 IEEE 32nd International Conference on Tools with Artificial Intelligence (ICTAI). IEEE Computer Society, 2020, where an auto-encoder based on graph convolutional networks (for both encoder and decoder) is trained to reconstruct both node features and the connectivity of the input graphs. However, being the encoder a graph convolutional network, it cannot directly deal with unseen nodes for which no connectivity is known.

The same issue occurs in Pan, Shirui, et al. “Learning graph embedding with adversarial training methods.” IEEE transactions on cybernetics 50.6 (2019): 2475-2487, which provides a structure-reconstruction term to regularize the latent space and make predictions of nodes.

In Wang, Daixin, Peng Cui, and Wenwu Zhu. “Structural deep network embedding.” Proceedings of the 22nd ACM SIGKDD international conference on Knowledge discovery and data mining. 2016, instead, both the encoder and the decoder do not make use of the structure either in the computation of graph embeddings or in the reconstruction of the node features, but the authors penalize their method to reconstruct the first and second order proximity of each node.

Song, Xuran, et al. “A graph-neural-network decoder with MLP-based processing cells for polar codes.” 2019 11th International Conference on Wireless Communications and Signal Processing (WCSP). IEEE, 2019 combines an MLP encoder with a graph ML decoder to address the problem of Polar Codes decoding. Here, it remains unclear how to reliably connect new unseen entities to the existing graph.

### SUMMARY

In an embodiment, the present disclosure provides a computer-implemented method of inductive graph machine learning, the method comprising: using information recorded or collected from an established network including a plurality of entities with relationships existing between the plurality of entities to generate a graph representation of the established network, wherein the plurality of entities form nodes of the graph representation and the relationships existing between the plurality of entities form edges of the graph representation; when a new entity arrives, mapping the new entity to a latent space; creating an extended network by connecting the new entity to one or more of the plurality of entities of the established network according to their distance in the latent space; and using the extended network and a graph machine learning (ML) predictor to make predictions about the new entity.

### BRIEF DESCRIPTION OF THE DRAWINGS

Subject matter of the present disclosure will be described in even greater detail below based on the exemplary FIGURES. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating an inductive graph machine learning method according to an embodiment of the present invention, instantiated for the specific use case of a patient network.

### DETAILED DESCRIPTION

In accordance with an embodiment, the present invention improves and further develops a method and a system of inductive graph machine learning in such a way that information given by relationships among the entities of an established network can be exploited for making predictions about new entities not yet connected to an established network.

In accordance with another embodiment, the present invention provides a computer-implemented method of inductive graph machine learning, the method comprising: using information recorded or collected from an established network including a plurality of entities with relationships existing between the entities to generate a graph representation of the network, wherein the plurality of entities form the nodes of the graph representation and the relationships existing between the entities form the edges of the graph representation; when a new entity arrives, mapping the new entity to a latent space; creating an extended network by connecting the new entity to one or more of the entities of the established network according to their distance in the latent space; and using the extended network and a graph machine learning, ML, predictor to make predictions about the new entity.

Furthermore, in accordance with another embodiment, the present invention provides a corresponding system and a tangible, non-transitory computer-readable medium as defined in the independent claims. Considering the structure-reconstruction term in the last function achieves the advantage that the known network structure can be explicitly exploited to make predictions.

According to embodiments, the present disclosure provides a method for inductive prediction of newly added (i.e. yet disconnected) entities to a given network, wherein the mapping in latent space is guided by a similarity-based structure-reconstruction loss. The proposed method allows to discover what is the “right” mapping of the new entity to its neighbors—and subsequently, in a second stage, exploit the structural information to solve a node prediction task. As such, the present disclosure provides a way to connect the new entity to others before making a prediction. By making predictions using the connections found (in latent space) between the new node and the existing ones, one is able to benefit from the additional structural information that already exists in the network. In all likelihood, this will lead to an improvement of the predictive performances of the system for the new entity, which is considered in isolation. In addition, by considering a new entity not in isolation, but in consideration of its closest neighbors in latent space, the amount of data-driven insights can be extended beyond what would be obtainable by just analyzing the features and target labels of such neighbors.

According to embodiments, a top-k similarity algorithm may be used to connect each new entity to the graph in latent space. A prediction for a new entity may be obtained by applying a graph-based ML method to the dynamically updated graph of entities.

According to a further aspect of the present disclosure, an interpretable method is provided that produces a list of entities deemed most useful for prediction by building a latent space according to adjacency information of the known graph, and applying a top-k similarity algorithm. The generic discovery of connections could provide a degree of interpretability for embodiments of the graph ML model disclosed herein, by shedding light on the subset of neighboring entities that mostly influenced a prediction.

According to an embodiment, the method may further comprise a step of generating, by a vectorial encoder prior to the creation of the extended network, a latent node embedding of the entities of the established network. The vectorial encoder may be implemented in form of a Multi-layer Perceptron, MLP, with a predefined number of layers and with Rectified Linear Units, ReLUs, as activation functions.

According to an embodiment, the vectorial encoder may be trained by applying a loss function that is configured to encourage a mapping of connected entities to similar latent representations.

According to an embodiment, the method may further comprise a step of computing, by a structure reconstruction module based on a distance metric between vectors, pairwise distances between the latent representation of the new entity and the entities of the established network.

According to an embodiment, the method may further comprise a step of using, based on the computed pairwise distances, a top-k similarity method or thresholds on similarity scores to connect the new entity to one or more of the entities of the established network.

According to an embodiment, the method may further comprise a step of providing, as an output in addition to a prediction about the new entity, a set of the closest neighbors of the new entity in latent space as a potential explanation for the prediction.

According to an embodiment, the method may further comprise a step of training the graph ML predictor by applying a supervised classification or regression loss function corresponding to a respective prediction task. In an embodiment, the graph ML predictor may be configured to use a Graph Isomorphism Network, GIN, as described, e.g., in K. Xu et al.: “How Powerful are Graph Neural Networks?” https://doi.org/10.48550/arXiv.1810.00826. In addition, the vectorial encoder may be further configured to receive training signals from the supervised classification or regression function corresponding to a respective prediction task. Accordingly, it may be provided that the vectorial encoder and the graph ML predictor are trained jointly (instead of being trained separately/sequentially).

According to an embodiment, the network may be a patient network including a plurality of patients, wherein patients' feature vectors include health related parameters of the patients. In this embodiment, the graph ML predictor may be configured to learn to make predictions about a heart disease severity level for a new patient.

According to an embodiment, the method may comprise a step of activating, in case the predicted heart disease severity level for the new patient exceeds a predefined threshold, an external device configured to initiate and/or provide for the execution of further diagnosis, e.g. additional blood tests.

According to an embodiment, the network may be a patient network including a plurality of patients, wherein patients' feature vectors include the patients' genomic activity information and information on the patients' response to a specific drug. In this embodiment, the graph ML predictor may be configured to learn to make predictions about a new patient's response to the respective drug.

According to an embodiment, the network may be a network including a plurality of districts of a city or a given area, wherein feature vectors of the districts include compound statistic of crime-related information, such as the number of minority classes and averaged ones like age, salary, and other features that may correlate positively and negatively with criminality. In this embodiment, the graph ML predictor may be configured to learn to make predictions about a criminality rate in a newly developed district (i.e. a district that is not yet connected to the existing network of districts).

There are several ways how to design and further develop the teaching of the present invention in an advantageous way. To this end, it is to be referred to the dependent claims on the one hand and to the following explanation of preferred embodiments of the invention by way of example, illustrated by the FIGURE on the other hand. In connection with the explanation of the preferred embodiments of the invention by the aid of the FIGURE, generally preferred embodiments and further developments of the teaching will be explained. In the drawing

Embodiments of the present disclosure provide a novel graph machine learning system and method that makes predictions about entities in a given network, as well as about new ones that are presented later to the network and for which no connectivity information is provided. The system connects the new entity to a number of existing ones according to their distance in latent space, and it outputs predictions of interest using the overall graph structure.

Basically, a naive approach to use when additional entities or nodes are provided would be to add them to the existing network and to retrain a graph ML model. However, embodiments of the present invention want to answer the question “what if we do not know how to add the new entity to the graph”? As discussed in the beginning, none of the relevant prior art methods takes into account the need to connect a new node to the graph. However, this is a very important use case in a number of applications, as it explicitly exploits the structure to provide predictions with an added degree of interpretability. The underlying assumption that applies to embodiments of the present disclosure is that the available features of the nodes of the existing network somehow suffice to determine—or at least approximate—the connectivity of the graph. Embodiments of the proposed method exploit this assumption to connect a new entity into the network before computing its prediction.

According to an embodiment, the present disclosure provides a method for inductive prediction of newly added entities to a given network, comprising the steps of
recording or collecting information (e.g., from a patient network) and transforming it into a graph (as described in detail in Section 1. below);
when a new entity arrives, mapping it to a latent space (as described in detail in Section 2. below);
connecting the entity in latent space to its most similar neighbors (as described in detail in Section 3. below), thus creating an extended network; and
using the extended network and the graph ML predictor to make predictions about the new entity (as described in detail in Section 4. below), and optionally returning the set of neighbors of that entity as a potential explanation for the prediction.

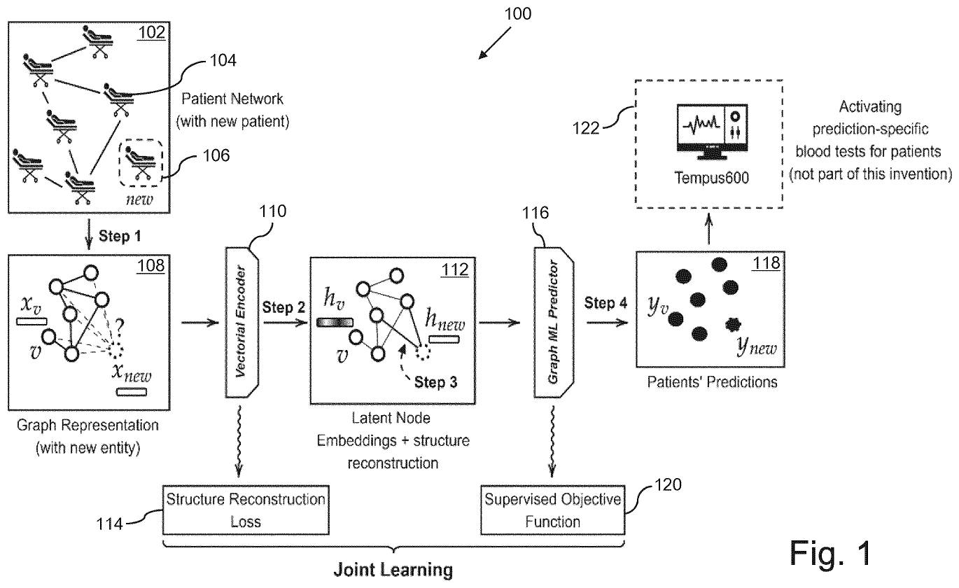

Embodiments of the present disclosure provide a graph machine learning (ML) system that is configured to make predictions about entities in a given network as well as about new entities that are integrated into the network at a later stage later and for which no connectivity information is provided. According to an embodiment, the system comprises i) an ML encoder for encoding the entities' features, ii) an entity that is in charge of connecting newly encoded entities to “reasonable” neighbors, iii) a graph ML predictor, and iv) an objective function that guides learning. FIG. 1 illustrates a respective embodiment, in which the above components/functions are arranged in a pipeline fashion. In the following, each component/function of this pipeline 100 will be described in detail. Without loss of generality and for ease of understanding, in the embodiment of FIG. 1, the pipeline 100 is exemplarily instantiated for a specific use case of a patient network 102 including a group of patients 104, e.g., patients admitted to a particular hospital, patients participating in a specific clinical study, etc.

### 1. Input and Problem Definition

According to embodiments of the present disclosure, it is assumed that a graph g is provided, which is extracted from a network, i.e. the patient network 102 in case of the embodiment exemplarily illustrated in FIG. 1. In this network 102, the network entities, i.e. the patients 104 in the illustrated embodiment, are connected with each other to form a graph g. The connection between the patients 104 may be established according to some procedure that may require data being available only at the end of a certain process, e.g. after medical history taking, hospitalization, treatment, or the like.

Generally, a graph g is a tuple (Vg, Eg, Xg), wherein Vg is the set of nodes forming the graph g (i.e. here patients 104), Eg is the set of directed edges (u,v) connecting pairs of nodes, and Xg is the domain of node features. xu∈Xg denotes the feature vector of node u. For instance, in the specific use case of a patient network 102, the feature vector xu of a patient 104 may include, e.g., measurements of the patient's 104 oxygen level, age, ethnicity, weight, blood type, etc.

As depicted in FIG. 1, when a new patient 106 is added to the existing network 102 (e.g., because the patient 106 is admitted to a respective hospital), it would be desirable to make predictions about the patient 106 by exploiting the additional information given by relationships among the patients 104 of the existing network 102 (i.e., g). For instance, in a concrete implementation, a task may be to predict a clinical risk for a new patient 106 and, depending on the predicted risk, to automatically activate an external machine, such as a sending station (e.g., a Tempus600®) to send small clinical samples directly to the laboratory to perform additional blood tests. However, because normally such connections can only be built at the end of the hospitalization process, there is no clear way to connect the patient 106 to the network 102.

It should be noted that building a graph by simply using a k-nearest neighbor approach based on patients' features is not feasible here, because the true relationships may not simply depend on the similarity between all the patients' features. However, it might be the case that a subset of these features—and/or their co-occurrence—allows to partially recover the true relationships between patients. This justifies an approach where the patients' features are first encoded in some latent space and only then similarity-based approaches are applied.

It should also be noted that the requirement that xu is a vector is not restrictive. If information is multi-modal, each data modality can always be mapped into a vector by a suitable (possibly pre-trained) encoder, and the resulting vectors can be stacked to obtain xu.

### 2. Features' Encoder

After having established the graph representation 108 as described above and as shown as outcome of step 1 in FIG. 1, in a next step, the features are encoded by an encoder 110. The encoder 110 can be any learnable ML model that, as shown at step 2 in FIG. 1, takes each vector of features xu as input and produces some latent vectorial representation hu of dimension d, where d is a hyper-parameter of the system. According to embodiments of the present disclosure, the encoder 110 may be implemented in form of a Multi-layer Perceptron (MLP) with 1 layers (with 1 being another hyper-parameter of the system), preferably with ReLU (Rectified Linear Units) as activation functions. The outcome of the feature encoding step (step 2 in FIG. 1) is the latent node embedding (yet without a structure reconstruction for the new patient 106) schematically shown at 112 in FIG. 1.

It should be noted here that the feature encoder 110 does not present any novelty per se, but its presence at this stage of the pipeline 100. In fact, according to embodiments of the present disclosure, the feature encoder 110 completely ignores the known structural information when encoding the entities in the latent space. This means that, when a new patient 106 arrives, the trained encoder 110 will seamlessly map the patient 106 in latent space. It is important to note that if one were to use a graph ML encoder to do so instead, the isolated patient 106 would have been considered as an out-of-distribution node, and the embedding would have not been consistent with those of the patient network 102.

### 3. Structure Reconstruction

According to embodiments of the present disclosure, the pipeline 100 may further comprise a structure reconstruction module 114. This module may be activated when a newly encoded patient 106 who does not (yet) belong to the patient network 102 is considered. The structure reconstruction module 114 may be configured to first compute, based on some choice of distance metric between vectors, the pairwise distances between the latent representation of the new patient 106 and those patients 104 in the available network 102. As distance metric, for instance, Euclidean distance, may be used. Then, by using methods such as top-k similarity or thresholds on similarity scores, the new patient 106 can be connected to others in the network 102, as schematically illustrated at step 3 in FIG. 1. Thus, the new patient 106 is now part of the network 102 and structure-aware predictions can be performed.

### 4. Graph ML Predictor

According to embodiments of the present disclosure, the pipeline 100 may further comprise a graph ML predictor 116 that is responsible for making predictions about nodes/entities of the network 102 (i.e. about individual patients 104, 106 in the illustrated embodiment) or about the entire graph, conditioned on the original and latent information of the nodes (see Section 2. above). In contrast to an MLP (Multi-layer Perceptron), a graph ML predictor may be configured to also take into account the structure of the network to make more informed predictions. Even though the proposed method is not restricted to any in particular, in the embodiment described here, the Graph Isomorphism Network may be used to predict the clinical risk of the new patient 106, as shown in FIG. 1 at 118 as outcome of step 4.

### 5. Objective Function to be Optimized

According to embodiments of the present disclosure, the pipeline 100 may further comprise a functional module 120 configured to consider a supervised classification/regression loss that affects the graph ML predictor 116.

In an embodiment, a combination of two loss functions is considered that are functional to the success of the proposed pipeline 100. These two loss functions, which may be learned jointly, may be used to train the proposed pipeline 100 and are not applied during the inference phase, i.e. the loss functions are not applied in connection with an incorporation of “new” patients.

First of all, the pipeline 100 may be configured to consider a structure reconstruction loss that is tightly bound to the structure reconstruction module 114 described above in Section 3. In particular, the loss may be designed to encourage the ML architecture to map connected patients to similar latent representations. In other words, the proposed model may be trained to find a mapping (as described above in Section 2.) to a latent space where it makes sense to consider feature similarity as a proxy for connections. This loss will only affect the feature encoder 110.

This can be achieved by framing this sub-problem as a binary classification problem where the loss is binary cross-entropy: ‘0’ means that an edge does not exist, whereas ‘1’ means that the edge is present in the network 102. Without loss of generality, it can be assumed to compute a score of each potential edge as the sigmoid of the dot-product between pairs of nodes' latent representations. To generate negative edge samples, i.e., with ‘0’ as target score, it may be provided to randomly select a number of edges—the amount may be chosen by the user of the pipeline 100—that do not exist in the original patient network 102.

According to embodiments of the present disclosure, the second loss term is the supervised classification/regression loss that corresponds to the problem that is being tackled. In the present case, it may be assumed to have a classification loss for, e.g., the prediction of heart disease severity and a multi-label classification loss that indicates, e.g., which additional blood tests may be required. Therefore, the target value of the training dataset should contain information about clinical risks and blood tests may be required by doctors in the first place. Alternatively, if the type of blood tests to take can be uniquely determined by the clinical risk, there is no need to gather this kind of information at training time.

The predicted clinical risk of the new patient 106, as shown in FIG. 1 at 118 as outcome of step 4, can be used for activating prediction-specific blood tests for the new patients. For instance, as shown in FIG. 1, the predictions may be used as input to an automatic blood test pipeline 122, like Tempus600, which executes a restricted set of blood tests, whose results may then be incorporated in the respective patient's clinical report.

To conclude, the present disclosure provides, for the first time, a simple and general way to connect new entities to a known graph, in such a way that structure can be exploited to make predictions. Taken individually, the components of the proposed pipeline 100 are not novel, but as described above, in particular in Section 4, the main characteristic that distinguishes the proposed pipeline 100 from prior art solution, lies in the carefully designed interplay of components and loss functions to connect a new node to an existing graph, followed by the exploitation of the additional connectivity.

Embodiments of the present disclosure can be implemented as a key addition to the already established GraphAI technology. Generally, the inductive approach proposed herein may help in many use cases, some of them will be described exemplarily in the following. For all use cases mentioned herein, the pipeline 100 described above in connection with FIG. 1 may be implemented in an adapted fashion.

Use Case 1—Patient Network: Prediction of heart disease severity and associated blood tests.

Use Case: Predicting the heart disease severity level of patients allows for efficient and timely strategies to sensibly reduce the deterioration of illnesses and in some case prevent deaths. Usually, when doctors suspect that there are risks for a patient after preliminary screening, they request additional exams to be taken depending on the severity, for instance blood tests. When trying to automatize predictions of heart disease severity, patient networks have already been proved to be an effective strategy, as they incorporate patients' information and meaningful relationships between them. However, it is often not clear or easy to retrieve how these connections have been constructed; in other cases, the information used to construct such networks is available only after certain activities/events/processes, e.g. after a patient leaves the hospital. For this reason, when a new patient arrives, it is important to reasonably find similar patients from which to gather insightful information. This will allow for network-aware predictions of heart disease severity levels and an automatic execution of additional tests, e.g. by blood machines, to minimize the time spent manually scrutinizing the individual patient and maximize the reaction time should severe risk be predicted.

Data Source: In this embodiment, a patient network, e.g. patient network 102 discussed above in connection with FIG. 1, may be used as a data source to build the graph g. The patient network may include healthcare-related data of a number of patients, such as oxygen level, temperature, weight, average heart rate, blood pressure, and other metrics of interest.

Graph ML method according to the present disclosure: According to an embodiment of the present disclosure, the pipeline 100 may be configured to predict, when a new patient arrives, which is the severity and risk of a heart disease and the set of blood tests to take. First, it may be provided that the patient is mapped to a latent space, which will be used to connect it to the patient network. Then, the resulting network may be used to make predictions about the new patient.

Output: Prediction of heart disease severity levels, associated with a list of blood test exams to execute, for the new patient. In addition, according to an embodiment, the method may be configured to provide a list of patients that were deemed more closely related to the new patient for prediction.

Physical Change (Technicity): The output of the proposed system can be used as input to an automatic blood test pipeline, like Tempus600, which executes a restricted set of blood tests, whose results may then be incorporated in the respective patient's clinical report.

Use Case 2—Patient stratification: Predicting the response to treatment in clinical trials.

Use Case: One of the factors which mostly impact clinical trials' budget, and ultimately their outcome, is the patient selection process. Many times, patients who should not be eligible for a specific treatment (because the trial will not cause a positive response) are enrolled anyway, and this can cause a huge monetary loss that ultimately leads to the failure of the trial itself. The rationale is to perform a stratification of patients using information at a genetic level, to predict their response at an earlier stage of the trial: this will result in a stratification between responders and non-responders. Given a graph of patients and their response to a specific drug, wherein the patients are connected within the graph according to the distribution of their cell types, it may be desirable to predict a response for a new entity (patient), for which only a single modality is available (i.e. the single cell data). In the context of this embodiment, the assumptions are made that i) patients with similar distributions of cell types react similarly to the respective drug and that ii) the set of gene-cell information correlates with such distribution.

### Data Source:

In this embodiment, a network of patients may be used to build the graph g, wherein the network includes patients' genomic activity information (e.g., single cell RNA-sequencing), their response to a specific drug, and publicly available ontologies. For any new patient, only genomic data are needed.

Graph ML method according to the present disclosure: According to an embodiment of the present disclosure, it may be provided that a new patient will be inserted into an already existing graph of patients, thereby exploiting at least one of the above assumptions i) and/or ii). Based thereupon and following the pipeline 100 described in connection with FIG. 1, the response to the drug may be predicted in a completely inductive fashion by also looking at the most similar neighbors.

Output: Prediction of the patient's response to the drug.

Physical Change (Technicity): The response prediction may be inserted in an entity that performs the screening of candidates, e.g. a Clinical Research Organization system. According to embodiments, the response may be used to produce a verdict (e.g., responder/non-responder) that a trial manager can use to include or discard a respective patient.

Use Case 3—Public Safety: Criminality rate assessment in newly developed areas.

Use Case: An important issue of public safety lies in the intelligent distribution of resources, let it be drones, police staff or military, across different areas. More often than not, adjacent districts can have very different criminality rates, so one cannot simply assume that, when moving from one to another, an individual's level of safety will only slightly change. In regions with extremely high population density and a dramatic expansion speed, it is of paramount importance to quickly assess the criminality rate of the newly developed areas, in order to discourage potential criminality clusters and improve safety. Assuming that a graph of meaningfully connected areas has been provided, a system according to embodiments of the present disclosure can exploit this information when a new area is developed and populated in order to preemptively predict the criminality risk and deploy resources accordingly.

### Data Source:

In this embodiment, a network of districts may be used to build the graph g, wherein the data of the districts may include compounded statistics such as the number of minority classes and averaged ones like age, salary, and other features that robustly and strongly correlate (positively and negatively) with criminality.

### Graph ML Method According to the Present Disclosure:

According to an embodiment of the present disclosure, it may be provided that a criminality rate for a newly deployed area is predicted, assuming the rates for other areas are known. When a new area is populated, it is added to the network of districts using the information available in the static features. Then the network may be used to predict the criminality rate, following the correspondingly adapted pipeline 100 described in connection with FIG. 1.

Output: Prediction of criminality rate. This information can be used to re-normalize the distribution of resources across different areas. In addition, the method may return a list of districts that were deemed more similar to the new area when predicting the criminality rate.

Physical Change (Technicity): The output of this system can be used to automatically re-balance the distribution of available surveillance means, e.g. drones, across the different areas/districts by means of an external system. The system may be configured to command most drones to relocate to areas with higher normalized criminal rates and significantly fewer ones in low criminality areas, so as to allocate resources in a sensible way.

In the practical use cases described above, the assumption works that the process used to connect entities together is unknown or depends on information unavailable when a new entity is presented. However, it should be noted that this assumption does not hold in general. When it is clear how to connect a new node to the graph, the inductive graph ML method described herein has only little or even no use. In addition, for the use cases described above, the implicit assumption has been made that the structure has been built—or at least correlates—with the features contained in the nodes. This assumption may also not hold in general, and experts deploying a potential technology should be aware of this.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method of inductive graph machine learning, the method comprising:
using information recorded or collected from an established network including a plurality of entities with relationships existing between the plurality of entities to generate a graph representation of the established network, wherein the plurality of entities form nodes of the graph representation and the relationships existing between the plurality of entities form edges of the graph representation;
when a new entity arrives, mapping the new entity to a latent space;
creating an extended network by connecting the new entity to one or more of the plurality of entities of the established network according to their distance in the latent space; and
using the extended network and a graph machine learning (ML) predictor to make predictions about the new entity.

2. The method according to claim 1, further comprising:
generating, by a vectorial encoder prior to the creation of the extended network, a latent node embedding of the entities of the established network.

3. The method according to claim 2, wherein the vectorial encoder is trained by applying a loss function that is configured to encourage a mapping of connected entities of the plurality of entities to similar latent representations.

4. The method according to claim 2, further comprising:
computing, by a structure reconstruction module based on a distance metric between vectors, pairwise distances between the latent representation of the new entity and the plurality of entities of the established network.

5. The method according to claim 4, further comprising:
using, based on the computed pairwise distances, a top-k similarity method or thresholds on similarity scores to connect the new entity to one or more of the of the plurality of entities of the established network.

6. The method according to claim 1, further comprising:
providing, as an output in addition to a prediction about the new entity, a set of closest neighbors of the new entity in latent space as a potential explanation for the prediction.

7. The method according to claim 1, further comprising:
training the graph ML predictor by applying a supervised classification or regression loss function corresponding to a respective prediction task.

8. The method according to claim 1, wherein the established network is a patient network including a plurality of patients, wherein feature vectors associated with the plurality of patients include health related parameters of the patients, and
wherein the graph ML predictor is learned to make predictions about a heart disease severity level for a new patient.

9. The method according to claim 8, further comprising:
activating, in case the predicted heart disease severity level for the new patient exceeds a predefined threshold, an external device configured to initiate and/or provide for the execution of additional blood tests.

10. The method according to claim 1, wherein the established network is a patient network including a plurality of patients, wherein feature vectors associated with the plurality of patients include genomic activity information associated with the plurality of patients and information on a response of the plurality of patients to a specific drug, and
wherein the graph ML predictor is learned to make predictions about a response of the new patient to the respective drug.

11. The method according to claim 1 wherein the established network is a network including a plurality of districts of a city or a given area, wherein feature vectors of the districts include compound statistic of crime-related information, and
wherein the graph ML predictor is learned to make predictions about a criminality rate in a newly developed district.

12. A system for inductive graph machine learning, the system comprising one or more processes that, alone or in combination, are configured to provide for the execution of:
using information recorded or collected from an established network including a plurality of entities with relationships existing between the plurality of entities to generate a graph representation of the established network, wherein the plurality of entities form nodes of the graph representation and the relationships existing between the plurality of entities form edges of the graph representation;
when a new entity arrives, mapping the new entity to a latent space;
creating an extended network by connecting the new entity to one or more of the plurality of entities of the established network according to their distance in the latent space; and
using the extended network and a graph machine learning (ML) predictor to make predictions about the new entity.

13. The system according to claim 12, wherein the graph machine learning (ML) predictor is configured to use a Graph Isomorphism Network (GIN).

14. The system according to claim 12, further comprising:
a vectorial encoder configured to generate, by prior to the creation of the extended network, a latent node embedding of the plurality of entities of the established network, wherein the vectorial encoder is implemented in form of a Multi-layer Perceptron (MLP) with a predefined number of layers and with Rectified Linear Units (ReLUs) as activation functions.

15. A tangible, non-transitory computer-readable medium having instructions thereon which, upon being executed by one or more processors, alone or in combination, provide for execution of a method of inductive graph machine learning, the method comprising:
using information recorded or collected from an established network including a plurality of entities with relationships existing between the plurality of entities to generate a graph representation of the established network, wherein the plurality of entities form nodes of the graph representation and the relationships existing between the plurality of entities form edges of the graph representation;
when a new entity arrives, mapping the new entity to a latent space;
creating an extended network by connecting the new entity to one or more of the entities of the established network according to their distance in the latent space; and
using the extended network and a graph machine learning (ML) predictor to make predictions about the new entity.
